(19) United States
(12) Patent Application Publication
(10) Pub. No.: US 2010/0145252 A1
(43) Pub. Date: Jun. 10, 2010

(54) Prevention of indwelling device related infection: compositions and methods

(76) Inventor: Hans-Dietrich Polaschegg, Kostenberg (AT)

Correspondence Address: PANDISCIO & PANDISCIO, P.C., 470 TOTTEN POND ROAD, WALTHAM, MA, 02451-1914, US

(21) Appl. No.: 12/460,787

(22) Filed: Jul. 24, 2009

Related U.S. Application Data
(62) Division of application No. 10/769,961, filed on Feb. 2, 2004.

(30) Foreign Application Priority Data
Feb. 3, 2003 (EP) 03002292.5

(51) Int. Cl. A61L 15/44; B05D 3/00

(52) U.S. Cl. 604/6.16; 427/2.25

(57) ABSTRACT

Catheters used for medical treatment, e.g., hemodialysis are filled with a locking solution, usually heparin between treatments. To prevent infections, antimicrobial or antibiotic substances have been used as locking solution alone or in combination with antithrombotic substances. It has been found that these locking solutions are rapidly washed out from the catheter tip. The invention describes a thixotropic gel that can be used as locking solution. Beneficial substances, e.g., antimicrobial or antibiotic substances can be added to the gel. A preferred antimicrobial substance is taurolidin alone or in combination with salicylic acid or one of its salts.

### FIELD OF THE INVENTION

This invention relates to methods and compositions, which prevent biofilm formation on or near medical prosthetic devices in order to reduce patient infection related to indwelling devices. The invention further addresses the complete life cycle of medical applications of an indwelling device teaching means to reduce or prevent infection.

### BACKGROUND

Medical prosthetic devices are widely used in medicine to treat chronic medical conditions as well as providing acute medical care. It is well known that implanted medical devices are often colonized with microbes and patients with medical devices have higher risk of becoming infected than patients without implanted devices. A study published in 1995 examined 38 ex vivo catheters from patients receiving chemotherapy. All catheters were coated with Biofilm even those implanted for only a few days and even in patients without signs of infection had colonized catheters (Anaissie E, Samonis G, Kontoyiannis D, Costerton J, Sabharwal U, Bodey G, Raad I. Role of catheter colonization and infrequent hematogenous seeding in catheter-related infections. Eur J Clin Microbiol Infect Dis 1995; 14:134-7) A German study on silver impregnated catheters showed that bacteria could be found on tips of catheters explanted after only 6 days of use. The likelihood for finding colonized catheter tips increased with the number of days of use (Boswald M, Lugauer S, Regenfus A, Braun G G, Martus P, Geis C, Scharf J, Bechert T, Greil J, Guggenbichler J P; Reduced rates of catheter-associated infection by use of a new silver-impregnated central venous catheter. Infection 1999; 27:S56-60, German version: Infection 1998; 26:S61-6)

A new sub-study branch has developed in Microbiology to study Biofilm (Costerton J W, Stewart P S, Greenberg E P. Bacterial Biofilms: A Common Cause of Persistent Infections. Science 1999; 284:1318-22) The Biofilm specialty has adopted new methods of imaging and quantification techniques to study this form of microbial populations. These new techniques helped elucidate the sequencing steps of Biofilm formation and show how most infections in patients with indwelling medical devices can be traced to the Biofilm adhering to the device. In the last years scientists have discovered that microbes in a Biofilm are considerably different from the microbes in the planktonic form, which were the only microbes studied previously.

Many hospitalized patients often become infected while in the hospital for unrelated diseases. Hospital acquired infections are called nosocomial infections and the incidence of these infections has been growing rapidly in the advanced countries of the world. The increase is caused by several factors including the emergence of bacteria resistant to antibiotics and the increased use of invasive medical devices and procedures. Catheters account for approximately 50% of all nosocomial bloodstream infections resulting in ˜400,000 episodes of catheter related bloodstream infections (CRBSI) each year in the USA. These increase the average length of stay in the intensive care unit (ICU) by 20 days with a medium cost of approximately US$92,000 per patient and is the cause of death of 15 to 35% of patients in the ICU. (Ryder M., The Role of Biofilm in Vascular Catheter Related Infection. New Developments in Vascular Disease published by Physicians & Scientists Publishing Company. Vol. 2, Number 2 (Fall 2001)). In Germany the number of catheter related blood stream infection has been estimated to ˜11000 per year resulting in ˜1000-1400 deaths and 40000-60000 additional patient days in the ICU (Gastmeier P, Weist K, Ruden H. Catheter-associated primary bloodstream infections: epidemiology and preventive methods. Infection 1999; 27:S1-6. The German version was published a year earlier: Infection 1998; 26:S1-7.)

Devices that have the highest rate of nosocomial infection include various catheters including ports and peripheral inserted central catheters (PICC) lines used for administration of liquids to the venous system such as saline, electrolytes, medications, imaging enhancers, chemotherapy drugs, and parenteral nutritional products as well as hemodialysis and hemofiltration. Other types of catheter devices such as enteral feeding tubes, spinal catheters, various shunts, urinary catheters, peritoneal dialysis catheters, intratracheal tubes for assisted breathing as well as diagnostic monitoring arterial catheters also carry high risk of Biofilm formation.

Patients in Intensive Care Units (ICU) of a hospital are particularly at risk for nosocomial infection. Many of these patients have several invasive lines such as urinary catheters, central venous catheters as well as endotracheal tubes for assisted breathing. These acute devices are life sustaining. Patients are often in the ICU for 3 weeks or longer while being attached to various invasive life support devices. The 3 most commonly used devices in the ICU are venous catheters, urinary catheters and endotracheal tubes, which are inserted in approximately 60% of ICU patients. Various studies report that these devices cause greater morbidity, mortality, and longer length of stay in the hospital than all other causes combined. Costs attributable to device related nosocomial infections in the US range from approximately 4 to 12 billion US$ per year.

Other devices that are used chronically outside the hospital also carry increased risk of infection. A partial list of these devices includes pacemakers and pacemaker leads, heart valves, orthopedic devices such as artificial hips, implantable automatic defibrillators, artificial heart assist devices, implantable infusion pumps, drainage devices and implanted dental prosthesis.

Microorganisms or microbes (including bacteria) are the most abundant life form on earth. These microbes can be found on skin; in the air and on almost all surfaces. They are in food, water and in the mouths and other body orifices offices. However, microbes are not normally inside body tissue or cells because the healthy body has skin as a barrier to the outside world and an elaborate effective defense immune system. This immune system senses the presence of microbes, seeks them out, engulfs and kills them and finally disposes of the waste products. Living creatures such as mammals, birds, fish etc have evolved a complex system of defense to protect themselves against microbes. Microbes, which gain entrance to the body's tissue or blood supply and grow, are the cause of infectious disease and potential death. When medical devices are placed and reside in the patient, the host defense mechanism may be impaired or evaded.

William Costerton and colleagues from the Institute for Biofilm Engineering (Bozeman, Mont., USA) and others have published studies, which elucidate the growth and functioning of Biofilm in many environments including the medical field. Biofilm is ubiquitous in nature and often coexist with host mammals if the host creates a blocking mechanism for entry of bacteria into the inner body space. Indwelling medical devices often provide a protected niche within the host to create a blocked off space where the microbes can maintain viability. For example, immediately after implantation of a subcutaneous device, a collagen skin begins to form and envelope the device. This skin, called a “pocket”, becomes a barrier between the device and vascularized tissue. This barrier blocks the immune system's proteins and cells in the blood, from reaching the target microbes to eradicate them. The microbes can therefore proliferate without challenge. Consequently, small numbers of microbes, which were on the device at the time of implantation can grow and form quickly into Biofilm mass. Also, even if the immune system cells reach the device, the artificial surface of the device inhibits the body's immune system from attacking and eradicating attached microbes. Once the Biofilm is established, the microbes change in profound ways. Most significantly, they become much more resistant to the effects of bactericidal effects of antibiotics compared to bacteria in a planktonic form. Furthermore, many microbial life forms produce a gel matrix (called slime) as they transform into Biofilms. The slime matrix encasing the microbes is a further barrier that the antibiotics must penetrate to reach the microbes and kill them.

Biofilm can grow into a large mass. The Biofilm mass size is limited by the ability of the diffusion pathway to convey nutrients and carry away waste products from the microbes within the mass structure. When the pathway becomes overwhelmed with material, the mass stops growing. Implanted devices colonized with Biofilm microbes into the blood stream or into healthy tissue and cause infection. Because of the transformational change in the microbes within a Biofilm, the infection is very difficult to treat with the device implanted in a patient. In many clinical situations doctors decide that eradication of the infection is impossible without removal of the device. This can be a dilemma for the patient as often implanted devices are life sustaining. The risk of not effectively eradicating an infection must be weighed against the loss of function provided by the device. This is a common situation in hemodialysis treatment where catheter related blood stream infection is a high cause of death and device related infection is intractable to treat by conventional means. For these reasons, many doctors choose to remove infected catheters. Of course the patient still requires hemodialysis, which requires some type of blood access. So the patient is usually given a course of systemic antibiotics, the infected catheter is removed, a new catheter is placed in a new site and the patient is closely monitored to determine effectiveness of infection treatment. However, this is not a good long-term practice, because blood vessels often get damaged during catheter insertion and patients have only a few catheter placement sites.

Some patients do not experience an overt incident of infection even when the device is contaminated with bacteria. Patients with good immune systems can often resist bacterial assault over a considerable period of time. However, recent studies show that patient under chronic exposure to bacteria often exhibit of an increased inflammatory state as measured by C Reactive Protein (CRP) in their blood along with other markers. This condition is linked to an increase in cardiac complications and mortality. Also several other diseases are linked to chronic bacterial assault.

A further problem results from this infection. Biofilm is difficult to eradicate, requiring long-term antibiotic treatment for several weeks. Persistent long-term exposure of microbes to antibiotics imposes environmental pressure on microbes to evolve into a hardier resistant form in order to survive and reproduce. The emergence of bacterial resistance has become widespread and threatening to the health levels achieved since the use of antibiotics in the 1950s. Many countries of the world have initiated programs to reduce the use of antibiotics in animals and humans as a result.

Devices become colonized because microbes from the outside world penetrate the patient's microbial barriers. The skin is the major barrier between tissue, cells and body fluids. Openings in the body such as the mouth, nose and other body orifices incorporate various means of inhibiting the growth and passage of microbes into the inner space of the body. The surgical placement of a device into the body offer a major penetration opportunity for microbes to get inside. Even with the most rigorous aseptic controls, it is virtually impossible to prevent some numbers of bacteria from being carried into the body on outer surfaces of the devices. If the bacteria can evade the host immune system for a few hours and survive they will grow into a biofilm.

Catheters and other transcutaneous devices (i.e., devices which cross the skin barrier) carry an additional means for microbe entry. The interface between the catheters outer surface and the host tissue provides a tiny space allowing microbes to migrate into the body from outside. Additionally, the surgical procedure itself adds to the risk. Certain placement techniques tend to drag or push bacteria into the inner space of the patient. For example pushing catheters into a blood vessel using a non-cutdown surgical method may allow the catheter to rub across the patient's skin prior to entry into tissue and the blood vessel and carry in microbes from the patient's own skin. It is virtually impossible to remove all bacteria from a living patients skin.

Another example is the insertion of a urinary catheter into the urinary bladder. It must pass through the Penis (or Vagina) and into the Uretha which are known to harbor high levels of microbes. Similarly, during the insertion of Ventilation tubes, the device has considerable contact with the Mouth and Trachea, which are highly colonized with microbes.

Subcutaneous implantation of devices such as pacemakers, implantable pumps and ports, create a sanctuary for bacteria to thrive in the subcutaneous pocket encapsulating the device. The pocket isolates the device from the host tissue and blood supply to protect the host from the foreign materials and protects the microbes, which may have been deposited on the device providing a safe haven from the body's immune system.

A common but serious problem with catheters placed in the blood stream is that these devices are often used to withdrawn blood or administer blood products. Blood must flow to maintain its fluidity. In a stagnant blood pool a cascade of steps changes the blood from a fluid to a solid form (i.e., clot). So it is often the case that any blood that is left in a catheter during a time of no fluid exchange will clot. This thrombus may plug the catheter or at least fill a portion of the lumen with clot fragments causing an increase in flow resistance. It has been established that clot fragments are preferred sites for harboring microbes.

Standard catheter practice is to instill a catheter “lock” after blood flows through the catheter. The locking procedure consists of several sequential steps. Usually the lumen of the catheter is initially flushed with a small quantity of saline (i.e., normally 2 to 4 times the volume of the catheter's internal volume) to remove any blood occupying the internal space of the catheter. Then the catheter is instilled with a precise amount of liquid anticoagulant such as heparin or low concentration sodium citrate (i.e., 1 to 4%), which is nearly equal to the internal volume of the catheter. Then the catheter is clamped shut and the sealing cap on the proximal end is put in place tightly as a redundant seal. The fluid-sealed in the catheter is called a lock.

This catheter locking procedure is somewhat effective in reducing clotting compared with not using an anticoagulant in many applications. However, even with this anticoagulant lock, catheter complications of plugging and increased flow resistance are still common. The use of these anticoagulant locks does not prevent Biofilm formation within the catheter and does not prevent catheter related blood stream infection in the host.

Over the last ˜15 years several attempts have been made to prevent and/or to treat infections in patients related to the use of indwelling medical devices. In central blood catheters studies have been performed and patents issued teaching the use of locking solutions including a.) Comprising antibiotics agents, b.) Antibiotics mixed with anticoagulants, c.) Antimicrobials mixed with anticoagulants d.) High concentration citrate that acts as both an anticlotting agent and a bactericidal agent and e.) Ingredients comprising Taurolidine, citric acid and sodium citrate f.) Antimicrobial and anticoagulant locks with density and viscosity additives and g.) Alcohol mixed with anticoagulants. Although these locks have some merit they do not solve the twin problem which plague most catheters which is the very high complications resulting from clotting and infection. In addition some complications are caused by the locks.

It is now clear that the use of antibiotics as a prophylaxis carries a critical harmful consequence. Most infection control experts and regulatory agencies feel that widespread chronic use of antibiotics will hasten the loss of life saving antibiotics. In 2002 in the USA, strains of Staph aureous have been discovered in two unrelated patients, which are totally resistant to Vancomycin. This has had a frightening effect on the health system practitioners, as Vancomycin had become the drug of last resort as earlier used drugs lost their effectiveness against this common pathogen. As resistant bacteria make further gains, drugs may not be able to eradicate bacteria causing fatal diseases. For these reasons, experts say that the use of antibiotics should only be used in treating life threatening diseases rather than widespread application as a preventive measure.

Although some lock compositions used have improved the art, none has eliminated both clotting and Biofilm formation inside catheters. A major factor in this result is because a full strength lock solution is usually not achieved nor retained throughout internal volume of the catheter lumen during the time interval between uses. In an unintended action, a significant volume of the lock solution leaves the catheter. This action causes blood to enter the catheter resulting in the lock's concentration to fall below the minimum level necessary to prevent clotting of that blood. Also, the lower lock concentration fails to block attachment of bacteria that have entered the catheter during use and the formation of Biofilm Experiments show that the concentration of the lock solution injected into the catheter at the time of instillation becomes greatly diluted in the distal portion of the catheter. This result is primarily caused by 3 different factors as defined below:

During instillation of the lock solution into a catheter; typically with a syringe; the lock solution flows according to well-understood principles in fluid engineering. Normal fluids flow in either a “laminar flow” or “turbulent flow” regime. In a manual syringe instilled lock using typical lock liquids, the flow is typically in the laminar flow regime. In laminar flow, the flow streams (or particles) in the tube, flow in parallel lines in the direction of the center axis (i.e., flow lines do not mix) and the flow velocity is maximum at the center of the tube and near zero (i.e., stagnant flow) at the wall of the tube. At points between the center and the wall, the flow velocity is between zero and maximum velocity following a well-known engineering formula, which is a parabolic curve (Nichols WW, O′Rourke M F. The nature of flow of a fluid. Nichols W W, O′Rourke M F, editors. McDonald's Blood Flow in Arteries. Edward Arnold, A division of Hodder&Stoughton, 1990:12-53). This means that locking solution will be leaving the center portion of the catheter lumen well before fluid near the walls has reached the end of the tube. The inventor has measured this effect in a typical hemodialysis catheter and saline and found that lock fluid will be exiting the distal tip even when only about half of the fluid needed to fill the lumen has been injected into the catheter (Polaschegg H D, Shaw Ch. Catheter locking solutions: safety and efficacy of composition and methods. ASAIO J 2002; 48:178). The consequence is that a significant amount of lock fluid (˜15%) is spilled into the patient when the amount instilled is equal to the volume of the catheter lumen, which is the conventional technique of instilling locks. Also, resulting average tip concentration across the full area of the lumen is considerably lower than the instilled concentration. To achieve a concentration of lock solution equal to the concentration instilled, significantly more lock solution volume (˜20%) needs to be instilled than the volume of the actual catheter lumen. This would of course result in considerable spillage into the patient. Further more, under various amounts of fluid instilled, the concentration of the lock solution at the inside wall surface has a considerable lower concentration than the concentration at the center of the tube although it is more important to achieve the effects of anticoagulation and biocidal effects at the lumen wall than in the center. The purpose of the lock is to prevent adherence of clots or microbes to the surface of the lumen.

Density differences between a catheter lock and the host blood will cause flow to take place between the catheter and the bloodstream. For example, in a vertical catheter filled with lock solution and immersed in blood and with the proximal end sealed tightly and the distal tip open and with a lumen lock density slightly higher than the blood, locking fluid will exit the lumen and blood from the blood stream will enter the lumen to make up for fluid leaving the catheter (Polaschegg H D, Sodemann K, Estabrook B. Investigation of the effect of catheter side holes on flow properties and outwash of the locking solution. Proceedings of the 2nd Symposium on Angioaccess for Hemodialysis. Vascular Access Society, Maastricht, 1999: 195). In vivo, with the patient changing orientation during the course of the day (i.e., standing and sleeping, etc) new conditions arise and flow distribution is reset to the new conditions. The shifting of fluid in and out ceases when the catheter reaches mixture of blood and lock in density equilibrium.

Tip geometry is different among the various commercially available catheters. Many catheters, especially dialysis catheters incorporate radial directed side holes near the distal end and other catheters do not have side holes. It has been observed that side holes speedup the mixing of blood and lock solution occurring at the distal end and it is thought that side holes may help anchor thrombus to the catheter by a mechanical interlocking making it more difficult to displace and achieve good flow rates. Nevertheless, in all catheters considerable tip mixing occurs due to dynamic fluid flow in blood vessels. Central blood catheters have their tips near the beating heart, which influences fluid dynamic forces and catheter motion. The forces driving blood/lock exchange is especially severe when the catheter tip resides in the Right Atrium of the Heart. The Right Atrium is the collection chamber to the heart. It is in constant motion accepting flow from 2 opposite directions (i.e., blood from the lower part of the body and from the upper part). Each pumping cycle of the heart (i.e., approximately 1 beat per second at rest) causes abrupt movement of the atrium as the Right Atrium fills and collapses. Blood rushes from the atrium to fill the right ventricle for each beat producing a pressure pulse of approximately −5 to +5 mm of water in the right atrium during the cardiac cycle. Movement oscillations and the pressure pulses cause further perturbation of the fluid conditions in and around the catheter producing flow in and out of the tip passages in the catheter.

This dilution reduces the active agents to that provide antimicrobial and anticoagulation action to the lock. There is no doubt that this dilution phenomena is occurring. In many dialysis clinics in Europe and North America, a heparin concentration of 5,000 units per mL is placed in the catheter. This is approximately 1000 times higher level than required to prevent coagulation in blood and considerably more than required for anticoagulation of the patient. Yet even at this high level, most catheters still show major clot development at the tip, which can be seen when the lock solution is removed from the catheter during the preparation for a new hemodialysis treatment (i.e., typically occurs every 2-3 days). To find out if the powerful anticoagulant heparin was spilling from the catheters, clotting time testing was performed on typical hemodialysis patients immediately after the lock instillation. Doctors were surprised at the finding that lock spillage does occur in significant amounts causing patient systemic clotting times to become elevated. It appears that most hemodialysis patients are inadvertently anticoagulated for several hours after a lock instillation when locked with heparin (Karaaslan H, Peyronnet P, Benevent D, Lagarde C, Rince M, Leroux-Robert C. Risk of heparin lock-related bleeding when using indwelling venous catheter in haemodialysis. Nephrol Dial Transplant 2001; 16:2072-4 and Moritz, M., Vats, A., and Ellis, D. Systemic Anticoagulation and Bleeding in Children with Hemodialysis Catheters. Pediatr. Nephrol (2003) 18:68-70).

Systemic injection of the lock solution had tragic consequences for at least one patient. Hemodialysis catheters are intentionally placed so that their tips are directly in the right atrium of the hearts. This patient was instilled with high concentration Citrate Lock Solution (i.e., ˜35%) leading to heart arrest. The lock solution is described in PCT Patent Application WO 00/10385 published 3 Mar. 2000. The product was commercialized in the USA without US Food and Drug Administration (FDA) approval and a death occurred soon afterwards. FDA issued a safety alert warning of the use of high concentration citrate as a lock in central bloodlines and the manufacturer recalled the product, which has not returned to market. FDA limited the concentration of citrate for any venous infusion procedures to 4%. Citrate is a powerful chelator of Calcium ions. The presence of Calcium ions is necessary for muscle contraction and is involved in the pacemaker electrical signal cascade in heart muscle contraction synchronization. It is thought that Citrate spilled out of the catheter into heart muscle. The citrate chelated calcium ions in the heart causing heart dysfunction and death.

This accident illustrates the importance of a medical safety principle incorporated in the European Medical Device Directive. The Directive requires that risk analysis be performed to demonstrate that the patients life is not at risk if a foreseeable fault or user error occurs. For example, a single fault would be a nurse inadvertently giving a patient a second dose of lock solution.

An antimicrobial lock (PCT Patent Application WO 00/01391) comprising Taurolidine as an antimicrobial agent, citrate as an anticoagulant and citric acid as a stabilizing agent was evaluated in Hemodialysis patients with Ports and Catheters in a European Clinical Trial. Excellent results were reported for reducing catheter related infection nearly to zero (Sodemann, K., Polaschegg, H., Feldmer, B., Two Years Experience with Dialock and CLS (A New Antimicrobial Lock Solution), Blood Purif, 2001; 19:251-254). In a subsequent controlled study using the locking solution, which measured both infection and thrombosis, the results again showed substantial reduction in catheter related blood stream infection compared to conventional heparin lock. However, they also observed 3 times more patients had flow complications with the taurolidine lock than with a conventional heparin lock (Allon M. Catheter Lock Solution (CLS) for Prophylaxis of Dialysis Catheter-Related Bacteremia (CRB): A Pilot Study. J Am Soc Nephrol 2002; 13:34A)

Taurolidine is a molecule having several properties, which make it uniquely suited to be an excellent antimicrobial agent acting in or around the local environment of in vivo medical devices (Polaschegg HD. Taurolidine, a new antimicrobial catheter lock solution. Dialysis Times 2000; 7:1,8). These properties are:

A long history of safe use in several European countries. It has a high margin of safety if ingested or injected. It is approved for use as a peritoneal lavage for treatment of peritonitis and as a drug for treatment of osteomyletis
Broad spectrum effect on killing bacteria and is also effective in killing certain fungi associated with medical device infections
No reported evidence has ever been published showing the emergence of bacterial resistance
Large overdoses into the systemic circulation do not produce a pharmacological response
Relatively small molecular size for high diffusion rates through the slime gel encasing the
Biofilm microbes
Inactivates endotoxins released by dead bacteria (i.e., present in dead bacteria)
Taurolidine has other properties that detract from its capability as a local antimicrobial agent. These are:
The minimum inhibitory concentration (MIC) value of Taurolidine necessary to kill bacteria and fungi is much higher than that of antibiotics used in medicine. The saturation concentration of Taurolidine in water (i.e., without precipitation occurring) is not very much higher than the MICs.
Taurolidine needs to be in contact with microbes several minutes to several hours to achieve high kill ratios compared to disinfectants which kills microbes in seconds to a few minutes
Water is the preferred solvent for Taurolidine and/or Taurultam

Catheters are commercially available that have coatings applied to protect against antimicrobial infection. Most of the coatings contain antibiotics, which is not a good choice because it contributes to the steady emergence of bacterial resistance. Also, the coatings offer antimicrobial protection only for a for very short time span as they are used up typically in 10 5-20 days. This is insufficient for chronic catheters as, e.g., used for dialysis, TPN, and urinary catheters for nursing home patients.

There remains a need for methods and compositions for prevention of thrombus and Biofilm formation in fluid delivery systems and other indwelling medical devices. The need grows more critical as more invasive devices and medical procedures are used while the emergence of bacteria resistant to antibiotics accelerates.

### BRIEF DESCRIPTION OF THE DRAWINGS

These and other objects and features of the present invention will be more fully disclosed or rendered obvious by the following detailed description of the present invention, which is to be considered together with the accompanying drawings wherein like numbers refer to like parts and further wherein:

FIG. 1a illustrates a test apparatus which may be used to test thixotropic properties of a gel;

FIG. 1b illustrates a three-way cock of the test apparatus of FIG. 1a, wherein the three-way cock is shown in a “pressure holding” position;

FIG. 1c illustrates a three-way cock of the test apparatus of FIG. 1a, wherein the three-way cock is shown in a “pressurize” position;

FIG. 1d illustrates a three-way cock of the test apparatus of FIG. 1a, wherein the three-way cock is shown in a “vent” position;

FIG. 2 illustrates a test for a 40% glycerin in water solution, wherein time is plotted on the X (horizontal) axis and pressure is plotted on the Y-axis;

FIG. 3 illustrates the measurement results for gel sample no2, wherein time is plotted on the X (horizontal) axis and pressure is plotted on the Y-axis;

FIG. 4 illustrates the second half of the measurement results shown in FIG. 3;

FIG. 5 illustrates a hydraulic setup which was used for the third series of tests; and

FIG. 6 illustrates the result of the test measurement with the Glatt-Look gel diluted 1+2, wherein time is plotted on the X-axis and pressure is plotted on the Y-axis.

### SUMMARY OF THE INVENTION

The inventor has found that several new elements may be used to particular advantage in combination with Taurolidine and/or Taurultam to prevent clotting and Biofilm formation or the elements can be combined with other antimicrobial agents. In accordance with the present invention a composition and method is used to prevent the attachment and subsequent growth of Biofilm on surfaces along with the prevention of clotting in passages of indwelling medical devices. These elements separately and particularly in combination make a more functionally ideal catheter locking material, which solves the unmet need for a better catheter lock solution.

One embodiment of the present invention is a gel with thixotropic properties to keep the lock inside the catheter and not spill out during the time interval between uses. This is accomplished by making a hydrogel matrix as a drug delivery vehicle containing a biocompatible antimicrobial agent alone or with another active agents, which may be useful for particular purposes. The hydrogel matrix is biocompatible and, biodegradable in the bloodstream. The matrix can be a hydrogel (e.g., pectin, gelatin, etc), a protein (e.g., collagen, hemoglobin, etc), a colloidal substance (e.g., serum albumin etc), an emulsion or other adjuvant. Preferably, the matrix shall have structural integrity and be thixotropic while instilled in a catheter during its locking function, which may be for several days to a few weeks. Thixotropy is a property, which is exhibited by certain gels. It is a property characterized by a solid or semisolid substance that when shaken, stirred or subject to high shear forces becomes fluid like and can flow and then returns to the semisolid state when the forces and/movement are stopped. Alternatively, the gel could have the properties similar to that of the colloidal dispersion similar to a sauce called “Ketchup” (i.e., tomato sauce), which resists movement, or flow until a high shear force is imparted to the fluid and then it flows easily. It has been observed that certain thixotropic hydrogel substances can be easily injected into a catheter and the gel does not have the variable velocity exhibited with the parabolic velocity profile of a laminar flowing liquid. Instead the semisolid gel moves into the lumen and travels through it as a cohesive rod-shaped mass of material. The result is that one can completely fill the catheter with this gel and obtain a uniform concentration without dilution near the tip without the overfilling and corresponding dumping of lock into the patient.

This gel is retained in the catheter even when the catheter tip is flapping (i.e., similar to a flag moving rapidly in the wind) in the right atrium or subject to density differences or being subjected to moderate pressure variation pulses. However, the gel can easily be withdrawn from the catheter with a syringe. It has been observed that once a threshold level force is achieved the lock substance flows freely. The tendency to spill out and to mix with blood was greatly reduced during experiments. This substance achieves nearly a 100% lock concentration at the tip and will prevent the mechanical intermixing of blood in the catheter. In practice, the retained lock does not allow blood to enter the catheter. Accordingly, with the absence of blood, no clotting or thrombus formation will occur within the catheter. Other ingredients may be added to the gel matrix to provide further functional benefit. The preferred antimicrobial is Taurolidine, which can be added to the matrix as a micro particle powder, or encapsulated in liposomes, microspheres, or nanospheres. It should be appreciated that use of a thixotropic gel lock material, many different active agents and drugs including sterileants, lysing agents (such as Urokinase), imaging enhancers, catheter surface modifiers, antibiotics and antimicrobial chemicals can be formulated or mixed into the lock to provide additional functional characteristics and even drugs which can be delivered from the catheter lock a through diffusion mode through the catheter side wall. Mixing gel with a powder results in a saturated solution of the substance contained in the powder in the water phase of the gel with the remaining not-dissolved substance acting as a reservoir. As the dissolved substance leaves the gel by diffusion, additional substance is dissolved from the non-dissolved reservoir space. This is especially useful for long term application when the gel is filled into body cavities, e.g., pockets of implantable devices.

Hydrogel is a three-dimensional network with a large amount of water giving them good biocompatibility with material consistency that is soft solid-like with high diffusive properties to gases, chemicals and proteins. Most research on hydrogels from the beginning (Wichlere O., Lin D. Nature 1960; 185:117-8) has been geared towards biomedical applications.

A wide variety of biomedical applications has been developed since then including soft contact lens, corneal implants, ophthalmic antibiotics for dry eyes, drug delivery depots, artificial breast prosthesis, tissue engineering scaffolds, urinary catheter coatings, vaginal gels, wound-healing devices, injectable antibiotic forms for treating periodontal disease and skin moisturizers to name only a few.

Materials comprising hydrogels include natural polymers including serum albumin, collagen, or alginates as well as synthetic polymers such as polyvinyl alcohol, poly (ethylene oxide) or poly (hydroxyethylene) and polyelectrolytes, such as poly(acrylic acid), poly(styrene sulfonate), and carboxymethylcellulose (CMC). Serum albumin has antimicrobial properties, which would enhance an antimicrobial lock mixture. It is a material found in blood and is a useful viscosity adjuster.

The desired makeup of the hydrogel catheter lock is as a support material providing bulk cohesive integrity necessary (i.e., thixotropic characteristics) to retain the lock within the catheter between catheter uses. It is important that if a fault condition were to occur, wherein the gel is accidentally injected into the blood stream, the bulk gel will dissolve and not block flow in the small vessels of the blood stream. Hydrogel biodegradation is also important in other misapplications where the gel materials are only intended for short periods such as the controlled release of drug from a depot. For example, it is known that Hydrogels comprising boronic acid polymers and poly(vinyl alcohol) degrade in the presence of glucose (Lee S, Park K. Glucose-Sensitive Phase-Reverseable Hydrogels. Ottenbrite R, Huang S, Park K, editors. Hydrogels & Biodegradable Polymers for Bioapplication. American Chemical Society, 1996: chapter 2)

Upon further addition of water to a hydrogel formed from a hydrophilic polymer, polymer-polymer contacts are broken and individual polymer molecules are dissolved in water.

The first embodiment of this invention is to teach methods and formulation of a novel family of catheter lock materials that encompass a wide variety of active properties that fit the particular clinical application providing lock be retention within the catheter during the time the catheter is not being used. This embodiment is to provide a gel lock biocompatible, safe and thixotropic and comprising the active agent(s) which prevents clotting and Biofilm formation within the catheter.

Another embodiment of the invention is a novel antimicrobial formulation, which combines Taurolidine with Salicylic acid or Sodium Salicylate in an aqueous solvent. Salicylic Acid and Sodium Salicylate are drugs that have been used with antibiotic locks in catheters to enhance the biocidal action of the antibiotic alone and to inhibit the attachment of microbes to surfaces. This last attribute is especially important because the initiation of a Biofilm expression and growth require that the individual bacteria must first attach themselves to the underlying surface. By stopping attachment, Biofilm formation is blocked.

Salicylic acid and Sodium Salicylate are classified as analgesics and Non Steroid Anti-Inflammatory Drug (NSAID). They are used to treat pain and fever, swelling, and joint pain of arthritis or rheumatism and are available as prescription and non-prescription drugs and are combined with other agents to make up drugs.

Sodium salicylate has been demonstrated to have remarkable antibacterial activity, including the ability to enhance the activities of certain antibiotics. This drug inhibits adherence, growth and Biofilm formation. A study indicates that the efficacy of Vancomycin against Staphylococcus epidermidis is significantly enhanced when salicylate is added. Staphylococcus epidermidis is a common etiologic agent of catheter related infection. (Polonio R E, Mermel L A, Paquette G E, Sperry J F. Eradication of biofilm-forming Staphylococcus epidermidis (RP62A) by a combination of sodium salicylate and vancomycin. Antimicrob Agents Chemother 2001; 45:3262-6)

The inclusion of 5 milli Molar salicylic acid (SAL) in a medium inhibited both growth and Biofilm production of Staphylococcus epidermidis by up to 55%. This effect was determined to be a reduction in the production of Biofilm rather than a reduction in the bacteria before formation of Biofilm. (Muller E, Al-Attar J, Wolff A G, Farber B F. Mechanism of salicylate-mediated inhibition of biofilm in Staphylococcus epidermidis. J Infect Dis 1998; 177:501-3)

Slime-inhibiting compounds including salicylic acid and other NSAID inhibit the growth of microorganisms on catheters and other medical devices. Coating silicone rubber catheters with salicylic acid makes them resistant to bacterial attachment which supports the concept of preventing Biofilm formation and preventing catheter related infection (Farber B F, Wolff A G. Salicylic acid prevents the adherence of bacteria and yeast to silastic catheters. J Biomed Mater Res 1993; 27:599-602).

A further embodiment is that achievement of prophylaxis of device related infection must encompass the entire life cycle of the device in proper manner to be effective. Although this may seem obvious, it is not being practiced in hospitals and clinics around the world. This embodiment of the invention envisions that effective methods must protect against contamination and loss of sterility of the device during storage, while removing the device from its package, during placement of the device into the patient and while operating and maintaining the device in vivo. For example, Ventilator tubes for assisted breathing have a very high infection rate in the ICUs of Europe and the USA. They account for the highest mortality in the ICU due to nosocomial infection. The numbers of deaths and the cost of treating these infections are quite substantial as ˜60% of patients admitted to the ICU receive Ventilator breathing tubes. The infection that commonly occurs is called Ventilator Associated Pneumonia (VAP).

Endotracheal tubes are often installed in the ICU to patients who are sedated under conditions that are often less than perfect. During installation, the tubular device is inserted in the mouth and pushed down into the trachea to the respiratory tract. The insertion causes the tubular device to rub across surfaces of the mouth & oropharynx, which are heavily colonized with bacteria. Once the ventilator tube is in place, the cuff is inflated to make a seal around the outside surface of the tube and the trachea. It is known that aspiration around this seal occur continuously as breathing cycles of the ventilator milk secretions around the cuff of the tube. Patient's stay in the ICU for an average of 8 days on a ventilator. During this time the patients remain sedated and cannot cough which otherwise helps to expel contaminates and bacteria from the throat and lungs. Bacteria in the mouth and throat can move down the outside of the ventilator tube or the inside surface of the throat and finally reach the alveoles causing pneumonia which is very difficult to treat successfully.

This embodiment of the invention teaches methods, which reduce the level of viable bacteria into the lungs during the installation of the device and during use of the device. Prior to inserting the Ventilator tube into the patient, the tube shall be copiously coated with a slippery antimicrobial hydrogel. Some of the antimicrobial gel coating will be transferred to the trachea as the tube is being inserted into place. Also, during the insertion procedure, a light spray of antimicrobial solution is directed ahead of the tubes distal end to help coat the trachea surfaces with antimicrobial solution or gel to lessen the amount of viable microbes being pushed into the airway passages. A further embodiment is to use Taurolidine as an antimicrobial to further deactivate resulting endotoxins, liberated from the dead or impaired bacteria.

A further improvement is the incorporation of an active antimicrobial delivery system built into the ventilator tube. This system is activated after insertion to deliver an antimicrobial solution, which bathes the ventilator tube's outer surfaces and the inner surface of the throat with a biocompatible antimicrobial agent. This stops viable bacteria from migrating along the tube into the lungs and causing pneumonia. A nurse can refill the delivery system reservoir periodically. The schedule of delivery of this bathing action is controllable and can be performed automatically. The type of mechanical or electrical apparatus, which is outside the body, is well known by medical device engineers.

Another device also used in the ICU, which has a high risk of contamination and patient infection, is the urinary catheter. This device regarding prophylaxis is analogous to the ventilator tube. It is also installed under poor conditions. It must pass deep into the body following a tract that is usually heavily colonized and the results are that most patients with urinary catheters in place for more than 6 days acquire a nosocomial urinary tract infection. Infection related to this device would be much improved by using methods of rigorous infection prophylaxis during insertion similar to that described for ventilator tubes. Additionally, the device can be protected from Biofilm colonization by providing an active delivery system during the time of use while inserted in the urinary tract.

Other devices can be improved by incorporation of an antimicrobial composition incorporated in to the material structure of the device are urinary continence devices, wound closure and healing devices and periodontal disease treatment devices.

### EXAMPLES

### Antimicrobial Effect of Taurolidine/Sodium Salicylate Solution

In bench experiments, the kill level in standardized planktonic microbes populations after exposure for 24 hours to various Taurolidine and Sodium Salicylate (SS) mixtures was measured. 3 different batches of Taurolidine/SS were made up as follows: 2% Taurolidine in an aqueous base with PVP (i.e., an expedient in commercial Taurolidine) was the starting formulation. This drug is commercially available in several countries of the European Union as: Taurolin 2% from Boehringer Ingelheim (Germany).

To this liquid was added powered Sodium Salicylate and 1 Normal Hydrochloric acid to achieve a final pH of approximately 6-7. These ingredients were mixed at a temperature of approximately 40° C. to aid in dissolution

The mixture was put into a clean vial and capped several weeks before sending to a laboratory for microbiology experiments.

A certified commercial laboratory performed the antimicrobial testing. The testing was a modification of the NCCLS Test Method “Methods for Dilution Antimicrobial Susceptibility Tests for Bacteria That grow Aerobically”: Approved Standard-Fifth Edition, NCCLS Document M7-A5 according to a pre-approved protocol. The test organisms used in the experiments were Staphylococcus aureus ATCC # 700788, Staphylococcus epidermedis ATCC # 27626, Enterococcus faecalis ATCC # 700802, Pseudomonas aeruginosa ATCC # 27313, and Candida albicans ATCC # 90029. The test batch solutions were tested in duplicate in undiluted and diluted state. Each sample was inoculated with the microorganism to achieve a final concentration of approximately 5×10⁸ CFU/ml (i.e., CFU is colony forming units). Measurement of organism concentration to determine kill was after 24 hours exposure and the results are presented as a mean log reduction in viable bacteria.

The make up of the 3 batches were as shown in the following table

TABLE 1
Test Lock Solution Formulations

| | Batch 1 | Batch 2 | Batch 3 |
|---|---|---|---|
| Taurolidine (Wt./Wt.) | 1.5% | 2% | 2% |
| Sodium Salicylate | 0.1 Molar | 0.1 Molar | 0.4 Molar |
| pH | 6.3 | 6.1 | 6.1 |
| PVP (Wt./Wt.) | 5% | 5% | 5% |
| Balance | Water | Water | Water |

The killing death rate results as expressed as a mean log reduction isare shown in the tables 2-4 for the 3 batches of the lock formulation.

TABLE 2
Mean Log Reduction in CFU
Batch I (1.5% Taurolidine & 0.1 M Sodium Salicylate)

| | Staph | Staph | Enterococcus | Candida | Pseudomonas |
|---|---|---|---|---|---|
| | aureus | epidermedis | faecalis | albicans | aeruginosa |
| No Dilution (100%) | >5.0 | >5.0 | 3.2 | 3.3 | >5.0 |
| 1:2 Dilution (50%) | >5.0 | >5.0 | 1.4 | 0.3 | >5.0 |
| 1:10 Dilution (10%) | 3.0 | 4.4 | 1.9 | No Reduction | >5.0 |
| 1:20 Dilution (5%) | 1.6 | 1.7 | 0.8 | No Reduction | No Reduction |

TABLE 3
Mean Log reduction in CFU
Batch II (2% Taurolidine & 0.1 M Sodium Salicylate)

| | Candida albicans |
|---|---|
| No Dilution (100%) | >4.7 |
| 1:2 Dilution (50%) | >2.6 |
| 1:10 Dilution (10%) | No Reduction |
| 1:20 Dilution (5%) | No Reduction |

TABLE 4
Mean Log reduction in CFU
Batch III (2% Taurolidine & 0.4 M Sodium Salicylate)

| | Candida albicans |
|---|---|
| No Dilution (100%) | >4.7 |
| 1:2 Dilution (50%) | >4.7 |
| 1:10 Dilution (10%) | No Reduction |
| 1:20 Dilution (5%) | No Reduction |

Catheter Lock Spillage Experiments

In a first test series it was demonstrated that powder mixed with a hydrogel remaines uniformly distributed in the gel.

In a second series of test it was shown that the gel moves plug like in the catheter lumen when injected and that it remains there after the end of injection even when the distal side is vented while the proximal side (tip) is in a water bath. By measuring pressure relaxation it was possible to demonstrate the thixotropic property of the gel.

In a third series of tests it was shown that the gel dissolves rapidly into particles smaller than 5 μm after injection into an aqueous fluid.

The gels used for the tests were:

GLATT LOOK Trend Effects Gel from Schwarzkopf (Germany), a hair styling gel
Ingredients: Aqua, PVP/VA copolymer, sorbitol, carbomer, PEG-40 hydrogenated, castor oil, triethanolamine, panthenol, PEG-75 lanolin, propylene glycol, benzophenone-4, disodium EDTA, perfum, DMDM hydantoin, methylparaben, CI17200 and
ALOE VERA gel from Fruit of the Earth (USA). Ingredients: Aloe Vera gel, triethanolamine, tocopheryl acetate, carbomer 940, tetrasodium EDTA, DVDM hydantoin, diazolidinyl urea.

The powder paint used for one test series was DRYLAC RAL3000 product number 029/30420 from Tiger-Lacke, Wels, Austria.

Dry water soluble color Basovit red 425E from BASF Germany and Blue Food Color distributed by Smart&Final (USA) containing food color (blue 1), citric acid and benzoate of soda were used for colorizing the gels.

All tests were performed at room temperature

Test Series 1

Powder paint was added to the colorless Aloe Vera gel and stirred. The gel did not change color for a week. No settling was observed. This was the case for the undiluted gel as well as for dilutions 1+1 and 1+2.

The same powder paint was added to saline and to 40% glycerol solution. In both cases the powder settled within minutes to hours after stirring.

Adding water soluble color to the gel samples resulted in uniform colorization after stirring.

Test Series 2

Colorized water and 40% colorized glycerol solution was used as a reference for the tests performed with dilutions of the GlattLook gel: Dilutions of 1+1 to 1+3 were found to work best.

For the test reported here 27.95 g of water and 0.03 g of Basovit red color were added to 13.98 g of GlattLook resulting in a total weight of 41.96 g. The materials were mixed with a magnetic stirrer until the material's color appeared uniform.

Test-catheters were produced from silicon tubing 2 mm i.d., 4 mm o.d., 30 cm long (Merck Eurolab Austria, now VWR art.no. 2283875) connected with a barbed female luer-lock.

The test catheters were first flushed with water with the help of a 50 mL (mL=milliLiter) syringe and then slowly filled with the test fluid. The tip was put in a water bath and the catheter was allowed to hang freely from a holder. As soon as the syringe was removed the colorized water and glycerol solution flowed from the tip and mixed with the water. The same happened with the gel diluted 1+3 but not with the gels 1+1 and 1+2.

When gels 1+1 and 1+2 were injected directly through the catheter into a water bath the gels emerged from the catheter in a worm like fashion

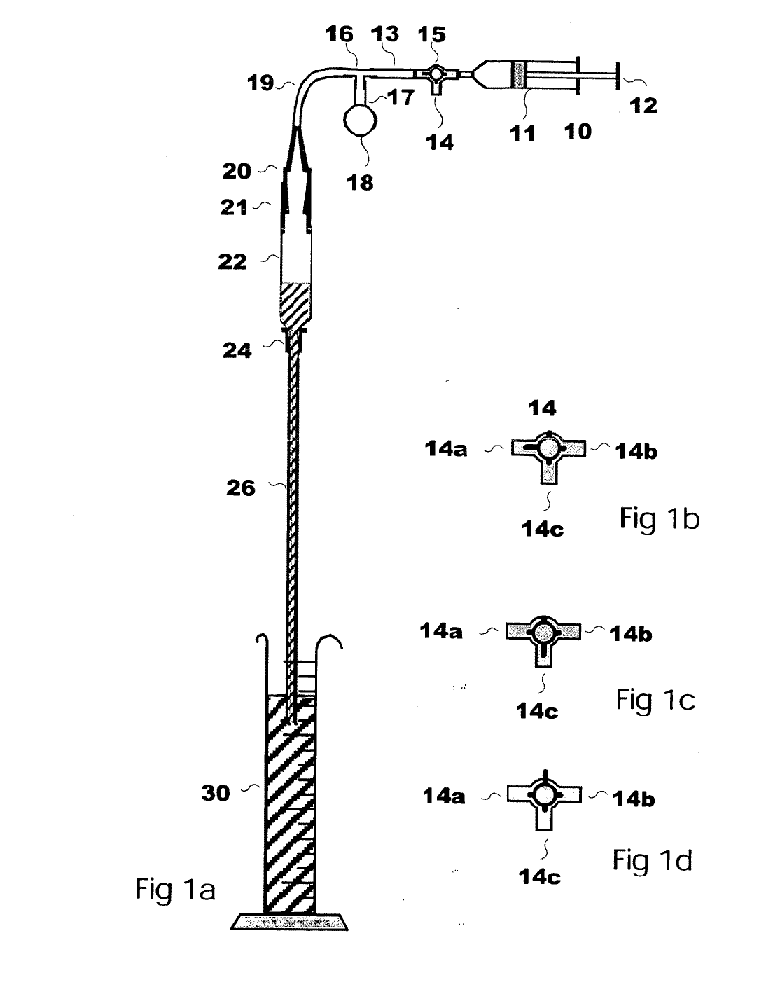

For testing the thixotropic properties the test setup shown in FIG. 1 was used. FIG. 1a shows the test setup. The body of a 5 mL syringe (22) is connected to the driving syringe (10) via a piece of silicon tubing, 12 mm o.d. (21), a barbed fitting (20), a piece of tubing 4.5 mm i.d, 6 cm long (19), a T (16), a piece of tubing 4.5 mm i.d. 3 cm long (13), a three way cock (15) to which the syringe 10 is directly connected. The syringe 10 consists of the syringe body (11) and the plunger (12). A piece of tubing 4.5 mm i.d. 15 cm long (17) branches from T (16) to a pressure transducer (18). Pressure transducer (18) is connected to a computer (not shown) for recording pressure versus time curves. The catheter (26) is connected to the syringe body (22) through a female luer-lock (24) which matches the male luer-lock of the syringe body (22). The catheter (26) hangs down in a S-like curve into a vessel (30) such that the tip of the catheter is below the fluid level in the vessel (30). The fluid level in the vessel (30) is approximately at the same height as the fluid level in the syringe body (22).

FIGS. 1b, 1c and 1d show the three positions of the three way cock. The three sides of the cock are (14a) which is connected to the syringe body (22), (14b) connected to the driving syringe (10) and (14c) which is open to the environment.

FIG. 1b shows the position when the syringe body (22) is separated form the driving syringe (10) and connected to the environment only through the catheter (26). This position is called “pressure holding” in the further description:

FIG. 1c shows the position of the cock when the syringe body (22) is connected to the driving syringe (10), called “pressurize” in the description.

FIG. 1d shows the position of the cock when the system is open to the atmosphere (“vent”).

For the test the empty catheter is initially not placed in the vessel (30) but into the container with the test fluid. The cock is positioned in “pressurize” (FIG. 1c) and approx. 3 mL of test-fluid (gel) is sucked into the syringe body (22) with the help of the syringe (10). The cock is switched into position “pressure holding” (FIG. 1b), the catheter tip is cleaned on the outside and put into the vessel (30) such that the tip is below the water surface. Next, the cock is put into position “vent” (FIG. 1d) and the system is allowed to equilibrate. Non-thixotropic fluids will flow out of the syringe body (22) through the catheter (26) into vessel (30) if the fluid level in (30) is below the fluid level in the syringe (22). For testing thixotropic gels the cock is put into position “pressurize” and the pressure increased to approximately 30 mmHg with the help of syringe (10). The cock is switched into “pressure holding” and the pressure decay is measured. During this process escape of filling fluid into the water space of vessel 30 is visually recorded.

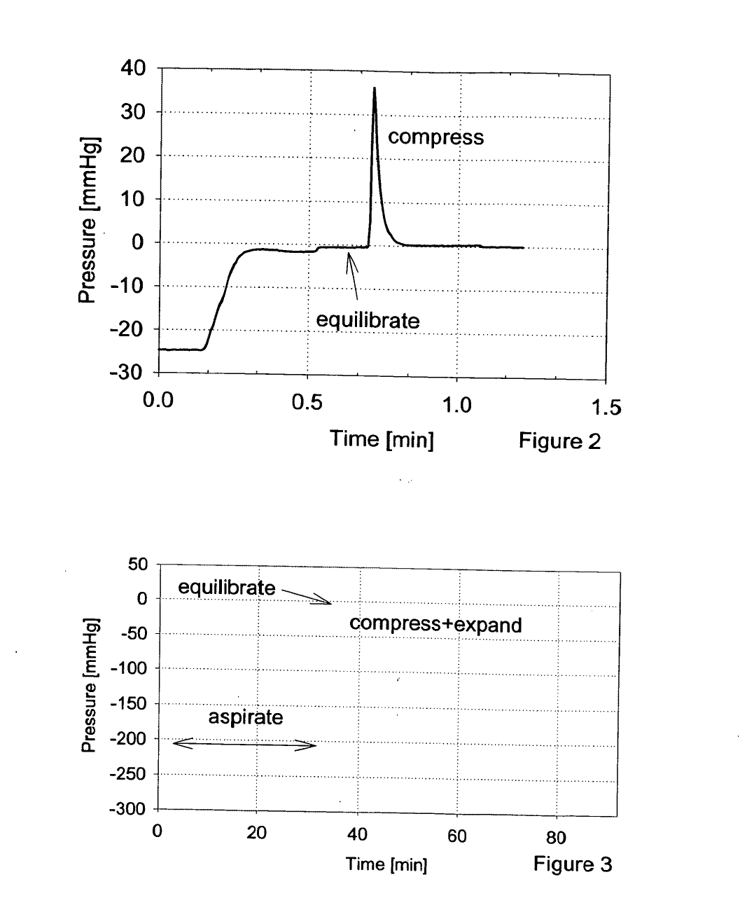

FIG. 2 shows the test for a 40% glycerin in water solution. The X (horizontal) axis is the time in minutes and the Y-axis is the pressure in mm of mercury measured by transducer (18). The dynamic viscosity of this solution at room temperature is approx. 3.6 mPa·sec which corresponds to the viscosity of blood at body temperature. The part of the curve between 0 and 0.5 [min] in the horizontal time scale shows the negative pressure followed by an equilibration phase during which the pressure is zero. The system is than compressed but the pressure equilibrates quickly when the glycerin solution is forced out of the syringe body (22) via the catheter (26) into the vessel (30). The process stops when the pressure in the syringe (22) is again zero.

FIG. 3 shows the measurement results for gel sample no2 1 part GlattLook+2 parts water. Initially the syringe (22) was put under negative pressure by retracting plunger (12) in syringe (10) which was then fixated in the final position. The pressure was then allowed to equilibrate slowly during the next 35 minutes while the test sample was aspirated. In order to bring the level in the syringe (22) to 3 mL the negative pressure was briefly increased which is visible as negative pressure peak in the tracing at approx. 35 min. After that the system was briefly equilibrated and the tip of the catheter was put into vessel (30). The system was compressed to approx. 40 mmHg, the cock was put into “pressure holding” position and the system was allowed to equilibrate while gel was flowing out of the syringe into the vessel (30).

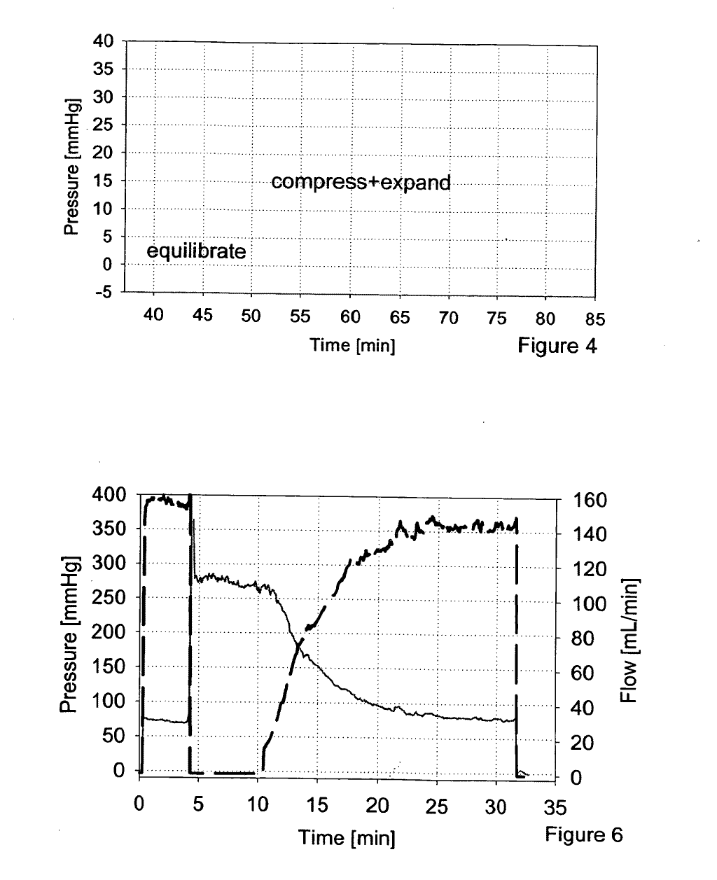

FIG. 4 shows the second half of FIG. 3. At 85 minutes the pressure in the system was 3.5 mmHg and no outflow could be observed from the catheter. Because the system was neither non-compliant nor absolutely air tight, the observed final pressure is a lower limit for the minimum pressure for flow.

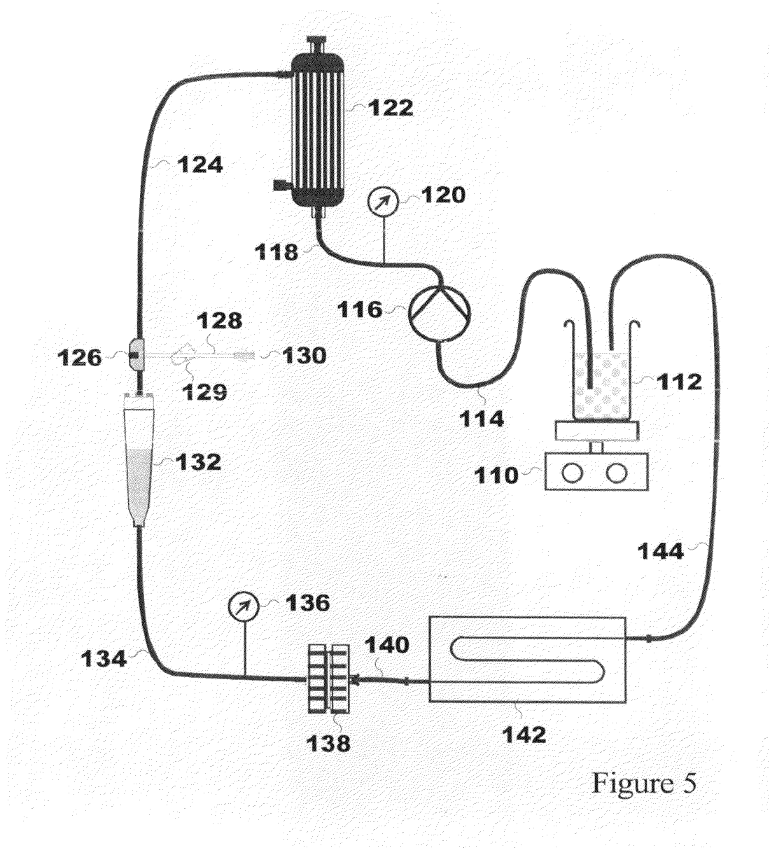

For the third series of tests the hydraulic setup described by FIG. 5 was used.

Water in a baker (112) is heated to 37° C. by a heater-stirrer (110). The gear pump (116) pumps water from the baker (112) through line (114) and line (118) to the pre-filter (122).

The pressure before the pre-filter is measured by the pressure transducer (120) which is connected to a computer (not shown). A high-flux dialyzer FX50 (Fresenius Medical Care Deeutschland) was used as pre-filter (122). The pre-filtered water leaves the filter (122) through line (124). The two other connectors of filter (122) are capped. Filtered water flows through the injection port (126) to drip chamber (132) and line (134) to the test filter (138). From the test filter (138) water flows through line (140) to the Coriolis flow meter (142) and back through line (144) to the beaker.

Injection line (128) branches from injection port (126). The injection line (128) can be clamped with the line clamp (129). Injection line (128) terminates with a female luer-lock (130).

From line (134) branches a line to “filter pressure” sensor (136).

Test filter (138) consists of a 25 mm filter holder (Sartorius AG, Germany:

Filtratisonsvorsatz 25 mm order no 16517) with a 5 μm mixed cellulose ester membrane filter (Advantec MFS, Inc. CA, USA cat.no: A500A025A). The flow meter (142) is a Coriolis flow meter from Danfoss (Denmark) type MASS2100 DI 3 with a low flow cutoff of ˜12 mL/min.

For the tests flow in the circuit was established with the help of a gear pump (116) which was operated at constant driving voltage resulting in a flow of approximately 150 mL/min. Initially the clamp (129) remained closed. The pressures and flow were continuously recorded by the computer. A 0.2 mL sample was aspirated into a 1 mL syringe. The syringe was connected to port (130) the clamp (129) was opened, the sample injected and the clamp (129) closed again. A 20 mL syringe filled with water was connected to port (130), the clamp (129) opened, the water injected and the clamp (129) closed again.

The result of the test measurement with the Glatt-Look gel diluted 1+2 shows FIG. 6. FIG. 6 shows the pressure measured by the “filter pressure” sensor (136) (left axis, full line) and the flow measured by the Coriolis flow meter (142) (right axis, dashed line) versus time. Flow is established for the first 4 minutes at ˜155 mL/min and the filter pressure id 75 mmHg. At 4.5 minutes 0.2 mL gel followed by 20 mL of water were injected as described above. Flow and pressure show a short peak and flow drops below the threshold of ˜12 mL/min and is indicated as zero in the tracing. Flow, however, could be observed at the outflow of line (112). Pressure increases after injection to ˜275 mmHg. After ˜7 minutes at time 11 min flow begins to increase and filter pressure begins to drop. Both approach the original value at time 25 min. Because the water used was not free of small particles the base line was not achieved. This has been found out during tests performed with water only.

Interpretation of Test 3:

In clinical practice it may happen that gel gets injected. It will be blocked by the lung. As the test shows the gel will dissolve within minutes into particles smaller than 5 μm which will not block capillaries in the circulation before they are finally dissolved and metabolized. The injected amount was only 10% of the amount that may be injected erroneously. The surface area of the lung capillary bed, however, is more than a factor 1000 larger than the surface area of the test filter.

1. A composition comprising a pharmaceutically acceptable gel containing an antimicrobial agent used for catheter protection

2. A composition as claimed in claim 1 wherein said gel is thixotropic

3. A composition as in claim 1 and claim 2 wherein said gel is a hydrogel

4. A composition as in any one of claims 1 to 3 wherein said gel is a microgel

5. A composition as in claims 1 to 4 wherein the antimicrobial agent is taurolidine, taurultam or a mixture thereof

6. A composition as claimed in claims 1 to 5 wherein said gel also contains an medically acceptable anticoagulant agent

7. A composition as claimed in any of the claims of 1 to 6 wherein said composition contains salicylic acid or one of its salts.

8. A composition as claimed in any of the preceeding claims wherein said gel can be injected into an indwelling catheter and said gel can be withdrawn from a catheter using conventional medical syringes.

9. A locking agent for an indwelling catheter that is composed of a thixotropic gel or a colloidal fluid that is retained in the catheter with minimal loss during instillation and/or the duration between uses of the catheter and can be instilled and withdrawn using a syringe

10. A catheter lock solution according to claim 9 in which the agent is albumin.
